(19) United States
(12) Patent Application Publication
(10) Pub. No.: US 2010/0028298 A1
(43) Pub. Date: Feb. 4, 2010

(54) INTERFERON TYPE I SUPPORTING COMPOUNDS

(76) Inventors: Mariela Bollati-Fogolin, Braunschweig (DE); Werner Müller, Braunschweig (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/444,266

(22) PCT Filed: Oct. 12, 2007
(86) PCT No.: PCT/EP07/08912

§ 371 Date: Apr. 3, 2009

Related U.S. Application Data
(60) Provisional application No. 60/851,049, filed on Oct. 12, 2006.

(51) Int. Cl. A61K 38/12; C07K 7/50; A61P 35/00; A61P 25/00; A61P 37/00; A61P 29/00; A61P 37/08; A61P 31/12; A61K 38/21

(52) U.S. Cl. 424/85.6; 530/317; 514/9; 424/85.4; 424/85.7

(57) ABSTRACT

The present invention relates to the use of cyclic peptides, in particular of vioprolides for the treatment and prevention of various diseases, disorders and conditions. In particular, the present invention provides compounds useful in enhancing and/or supporting interferon type I1 like interferon alpha or interferon beta, treatment or prevention of diseases, disorders or conditions. Further, the present invention relates to new pharmaceutical compositions comprising specific cyclic peptides, in particular, vioprolides, and type I interferon and its use in the treatment of various diseases, in particular, in the treatment or prevention of infectious diseases, cancers etc. Finally, the present invention provides methods for preventing or treating diseases, disorders or conditions susceptible to type I interferon treatment or prevention.

The present invention relates to the use of cyclic peptides, in particular of vioprolides fort the treatment and prevention of various diseases, disorders and conditions. In particular, the present invention provides compounds useful in enhancing and/or supporting interferon type I, like interferon alpha or interferon beta, treatment or prevention of diseases, disorders or conditions. Further, the present invention relates to new pharmaceutical compositions comprising specific cyclic peptides, in particular, vioprolides, and type I interferon and its use in the treatment of various diseases, in particular, in the treatment or prevention of infectious diseases, cancers etc. Finally, the present invention provides methods for preventing or treating diseases, disorders or conditions susceptible to type I interferon treatment or prevention.

### BACKGROUND OF THE INVENTION

Infectious diseases are the main cause of morbidity and mortality accounting for a third of the deaths which occur in the world each year. In addition, infectious agents are directly responsible for at least 15% of new cancers, and they also seem to be involved in the pathophysiology of several chronic diseases (e.g. inflammatory, vascular and degenerative diseases). The main strategies used to prevent infectious diseases are therapy and prophylaxis. Prophylaxis comprises inter alia vaccination or other preventive medicinal treatment of infectious diseases focussed on inhibiting viral replication in infected cells or reducing the number of copies of a virus in cells infected with the virus or other types of microorganisms or inhibiting infection of the cells. Interferons are a class of natural proteins produced by the cells of the immune system of most animals in response to challenges by foreign agents, such as viruses, bacteria, parasites but also by tumour cells. Interferons are one class of mostly soluble molecules known as cytokines.

Basically, there are three major classes of interferons, alpha interferon, beta interferon and gamma interferon. All of them display anti-viral and anti-antioncogenic properties, macrophage and natural killer lymphocyte activation, and enhancement of major histocompatibility complex glycoprotein class 1 and class 2. Further, interferons may direct T-cell response to a more TH1 phenotype by enhancement of differentiation of naïve T-cells to a TH1-phenotype and/or suppression of differentiation of naïve T cells to a TH2-phenotype. That is, interferon, like interferon alpha and interferon beta, can inhibit viral replication in virus infected cells—anti-viral activity—can influence the differentiation of T-cells—TH1 differentiation activity—or can inhibit cell proliferation—anti-proliferative activity. Generally, interferon alpha is secreted by lymphocytes like B- and T-cells, interferon beta is secreted by fibroblasts and interferon gamma is secreted by T-cells and natural killer lymphocytes.

In humans there are three major types of interferons:

1.
The human type I interferon consists of 13 different alpha isoforms, and single beta, omega, epsilon and kappa isoforms. Homologous molecules are found in many species, like rats and mice and in most other mammals. In addition, homologous have been found in birds, reptiles, amphibians and fish species. Other interferon types are described in various species including interferon beta, interferon nu, interferon tau and interferon delta. All type I interferons share the feature to bind to the interferon alpha receptor.
2.
The type II interferons consist of interferon gamma. Interferon gamma binds to the interferon gamma receptor complex and represents a typically member of cytokines indicative for a TH1-response.
3.
The human type III interferons encompass the recently described interferon lambda molecules comprising various isoforms.

Various pharmacological uses have been described for the different types of interferons.

Interferon-alpha has been shown to inhibit various types of cell proliferation, and is especially useful for the treatment of a variety of cellular or proliferation disorders frequently associated with cancer, particularly hematological malignancies such as leukemias. These proteins have shown antiproliferative activity against multiple myeloma, chronic lymphocytic leukemia, low-grade lymphoma, Kaposi's sarcoma, chronic myelogenous leukemia, renal-cell carcinoma, urinary bladder tumors and ovarian cancers. Further, interferon-alphas are also useful against various types of viral infections. Interferon-alphas have activity against human papillomavirus infection, Hepatitis B, and Hepatitis C infections. In addition, it is suggested that interferons and interferon receptors play a role in certain autoimmune and inflammatory diseases.

Today, interferon-alpha is an active ingredient in various pharmaceuticals for the treatment of hepatitis C or other type of viral hepatitis. In addition, interferon alpha is used for a chronic myelogenous leukemia. Interferon beta is presently the choice in the treatment and control of the neurological disorder multiple sclerosis. Further, it is described that interferon beta is able to inhibit the production of Th1 cytokines and the activation of monocytes.

Thus, various pharmacological uses for the different types of interferons have been described and claimed. However, various side-effects are known when administering interferons. In particular, adverse side-effects occur due to the action of interferon type I on the central nervous system. The most frequent side-effects are flu-like symptoms: increased body temperature, feeling ill, fatigue, headache, muscle pain, convulsion, dizziness, hair thinning, and depression. Further, dose-limiting toxicity, receptor cross-reactivity, and short serum half-lives significantly reduce the clinical utility of many of these cytokines. Thus, dosage of interferon administration is limited.

In the literature, combination of e.g. interferon alpha with other anti-viral drugs like ribavirin have been described.

Hence, there is still a need for new compounds and methods for the treatment or prevention or diseases, disorders or conditions susceptible to interferon treatment to enhance and/or support interferon type I activity.

From various bacteria, like Myxobacteria, secondary metabolites have been described having different capabilities and biological activities. For example, from myxobacteria a family of cyclic peptides is known, the so called vioprolides. Said cyclic peptides have been isolated from strain CB vi37 of Cystobacter violaceus as described e.g. in Schummer D. et al, 1996, Liebigs Ann. Chem. 971. They display some unusual chemical structures. In particular, some of the vioprolides have a 4-methyl-azeditine carboxyl acid replacing a proline in two of the described variants, a homoproline, replacing a second proline in two of the variants; and an L-glycerid acid interrupting the amino acid chain in one place. For the vioprolides anti-fungal and highly cytotoxic activities have been described.

The present invention addresses the need for further compounds which exhibit enhanced anti-viral, anti-proliferative and/or immune modulatory activities. That is, the present invention addresses to provide compounds useful in the treatment of various diseases, disorders and conditions involving interferon type I treatment. The invention provides new uses of compounds being effective to enhance interferon type I treatment in subjects suffering from various diseases, disorders or conditions susceptible to type I interferon treatment. Such molecules would be of beneficial use in a variety of applications, including e.g. therapeutic and prophylactic treatments, particular for viral infections such as HCV, HBV, HIV etc. and cancer, like leukemias but also in neurological disorders like multiple sclerosis. The present invention fulfils these and other needs.

### SUMMARY OF THE INVENTION

The present invention relates to the provision of the use of specific compounds or conjugates thereof, or salts or solvates thereof useful in therapeutic or prophylactic treatment of various diseases, disorders or conditions. In particular, said compounds are useful in enhancing and/or supporting interferon type I based treatment of various diseases like infectious diseases, cancer, tumours, autoimmune diseases, neurological disorders, allergies, or chronic or acute inflammatory processes, in particular, of viral-infections and leukemias.

The present inventors now found that cyclic peptides according to general formula I, e.g. vioprolides, have an interferon gamma type I enhancing and/or supporting activity. That means, the molecules according to the present invention are able to induce interferon type I in subjects and/or to increase interferon type I activity in subjects. Further, it is demonstrated in that compounds according to the general formula I have anti-viral activity, e.g. inhibiting replication of the virus or to protect subjects from viral infection. In addition, it is shown that the compounds according to the present invention have an anti-proliferative activity, thus, are useful in treating proliferative disorders like cancer and tumours.

Further, the present invention relates to new pharmaceutical compositions comprising the cyclic peptides according to general formula I and interferon type I. The combination of interferon type I with the molecules according to the present invention allow to reduce the dosage of interferon type I, thus, reducing the side effects associated with interferon type I treatment.

The pharmaceutical composition comprising vioprolides and interferon type I in effective amounts optionally together with pharmaceutically acceptable excipients can be used for the treatment or prevention of various diseases, disorders and conditions, in particular, infectious diseases tumours and cancer. Finally, the present invention relates to methods for preventing or treating infectious diseases, cancer or tumours, comprising administering the cyclic peptides of general formula I optionally together with interferon type I.

### BRIEF DESCRIPTION OF THE DRAWING

FIG. 1 shows a dose response curve for the cyclic peptide vioprolide A when combined with Interferon alpha tested in the system described in Bollati-Fogolin, M. and Müller W., J. Immunol. Meth., 2005, 306, 169-175.

FIG. 2 provides the results of flow cytometry analysis of the fibroblast cell line Mx-RAGE incubated with IFN alpha alone or a combination of IFN alpha and different types of vioprolides.

FIG. 3 demonstrates the increase of GFP positive cells by co-incubation with interferon type I and vioprolide A.

FIG. 4 shows the effect of vioprolide A on the IFN alpha and IFN beta standard curve demonstrating the enhancing or supporting effect of vioprolide A on interferon type I mediated pathways.

FIG. 5 demonstrates the anti-viral-activity of vioprolide A on VSV. The cytophatic effect was evaluated on MDBK cells. Different concentrations of rhIFN-α2b alone and in combination with 31 and 167 ng/ml of vioprolide A, respectively, were assayed.

FIG. 6 shows the anti-viral effect of vioprolide A on MSV-1. The cytophatic effect was evaluated on Vero cells. Different concentrations of vioprolide A were assayed, right side. Uninfected cells (left-most) and untreated Vero cells infected with 10⁴ PFU/ml HSV-1 (left) are shown for comparison.

FIG. 7: The antiproliferative effect of vioprolide A. The proliferation of WISH cells was tested at different concentrations of vioprolide A alone (grey) and in the present of IFN-α2b (black).

### DETAILED DESCRIPTION OF THE INVENTION

The present invention relates to the use of cyclic peptides according to the general formula I for the preparation of pharmaceuticals for treatment or prevention of infectious diseases, septic shock, cancer, tumors, or other proliferative disorders, autoimmune diseases, neurological disorders, allergies, or chronic or acute inflammatory processes. That is, the present invention relates to the provision of the use of specific compounds or salts or solvates thereof useful as enhancer of interferon (IFN) type I mediated treatment or prevention of various diseases, disorders and conditions. Preferably, said cyclic peptides of the general formula I is any one of the molecules also known as vioprolides A, B, C and D as shown below.

As used herein the term “disease”, “disorder”, “pathology” and “condition” relates to infectious diseases, septic shock, cancer, tumours, autoimmune diseases, neurological disorders, allergies or chronic or acute inflammatory processes. In particular, the diseases, disorders, pathology and conditions include but are not limited to viral infections, such as hepatitis B, hepatitis C, human immunodeficiency virus; bacterial infections, such as tuberculosis, leprosy and listeriosis, and parasitic infections such as malaria. Furthermore, preferred cancer and/or tumours include but are not limited to multiple myeloma, chronic lymphocytic leukemia, low-grade lymphoma, Kaposi's sarcoma, chronic myelogenous leukemia, renal-cell carcinoma, cervical carcinoma, urinary bladder tumors and ovarian cancers.

As used herein, the term “individual” or “subject” which is used herein interchangeably refers to an individual or a subject in need of the therapy or prophylaxis. The term “subject” or “individual” as used herein includes, but it is not limited to an organism; a mammal including e.g., a human, non-human primate (e.g. baboon, orangutan, monkey), mouse, pig, cow, goat, cat, rabbit, rat, guinea-pig, hamster, horse, sheep or other non-human mammals; a non-mammal including e.g. a non-mammalian vertebrates such as a bird or a fish.

The term “pharmaceutical composition” means a composition suitable for pharmaceutical use in a subject or an individual, including an animal or human. A pharmaceutical composition generally comprises an effective amount of an active agent and a carrier, including e.g. a pharmaceutical the acceptable carrier.

The term “effective amount” means a dosage or an amount sufficient to produce a desired result. The desired result may comprise an objective or subjective improvement in the recipient, i.e. a subject or an individual, of the dosage or amount.

A “prophylactic treatment” is a treatment administered to a subject or an individual who does not display signs or symptoms of a disease, pathology, or medical disorder, or displays only early signs of symptoms of disease, pathology or disorders, such that treatment is administered for the purpose of diminishing, preventing, or decreasing the risk of developing the disease, pathology or medical disorder. In this connection, it is noted that vaccination is one form of prophylactic treatment.

A “therapeutic treatment” is a treatment administered to a subject or an individual to display symptoms or signs of pathology, disease, or disorder in which treatment is administered into the subject for the purpose of diminishing or eliminating those signs or symptoms of pathology, disease or disorder.

The terms “interferon type I mediated treatment or prevention”, “interferon type I based treatment or prevention” or “interferon type I treatment or prevention” refer to a prophylactic or therapeutic treatment of a disease, disorder or condition wherein interferon type I is administered as an active ingredient of a pharmaceutical. Furthermore, the above terms comprise disorders, diseases or conditions wherein an interferon alpha activity allows treatment of said disease, for example, diseases where an up-regulation of interferon type I is beneficial. In this connection, beneficial means e.g. restoration of function, reduction of symptoms, limitation or retardation of progression of a disease, disorder or condition or prevention, limitation or retardation of detonation of a patient's condition, disease or disorder.

As used herein, the term “adjuvant” means substances which are added and/or co-formulated in an immunization to the active antigen, i.e. the substance which provokes the desired immune response, in order, enhance or elicit or modulate the humoral and/or cell mediated (cellular) immune response against the active antigen. Preferably, the adjuvant according the present invention is able to enhance or the innate immune response.

As used herein, the term “carrier” refers to a diluent, adjuvant, excipient or vehicle.

The present invention relates to the use of at least one of the cyclic peptides according to the general formula I

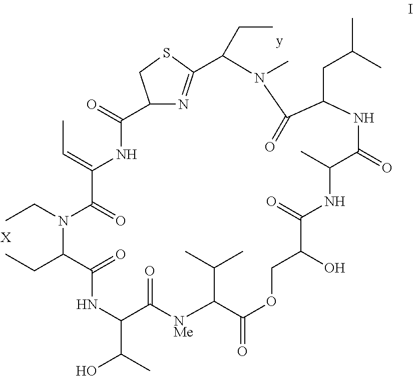

wherein X represents a methylene or an ethylene group and Y is an ethylene or a methylmethylene group,
or conjugates thereof, and salts or solvates thereof,
for the preparation of a pharmaceutical for the treatment or prevention of infectious diseases, septic shock, cancer, tumors, or other proliferative disorders, autoimmune disease, allergies, or chronic or acute inflammatory processes.

Preferably, the cyclic peptide is any one of the vioprolides shown below:

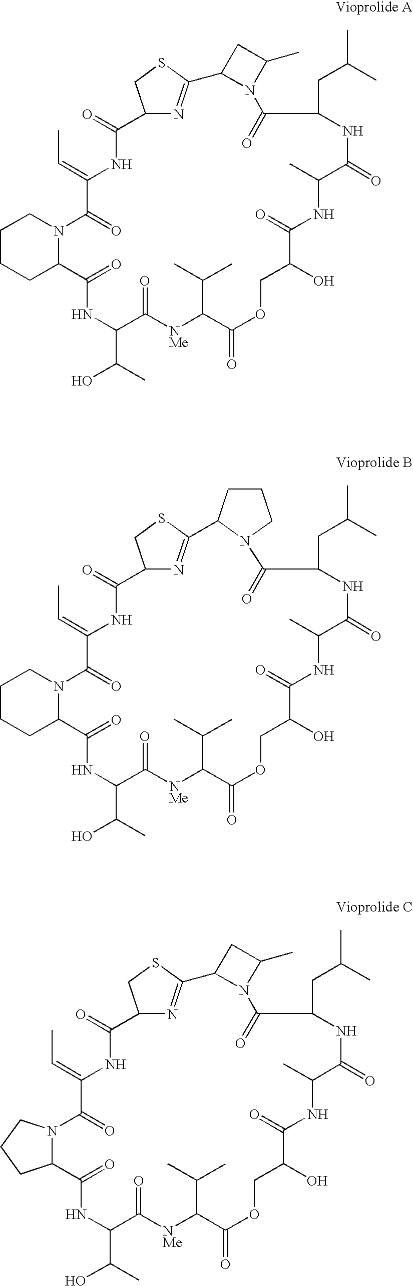

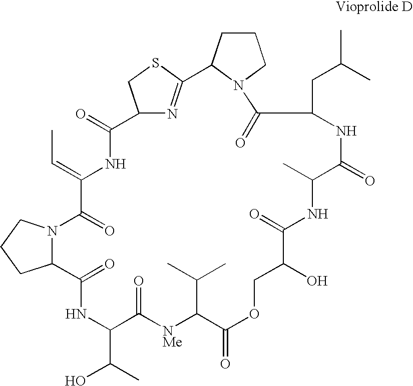

In a preferred embodiment the disease to be treated or prevented is an infectious disease. Infectious diseases include viral infections, such as Hepatitis B, Hepatitis C, Human Immunodeficiency Virus, bacterial infections, such as tuberculosis, leprosy and listeriosis, and parasitic infections, such as malaria. In particular, preferred compounds according to the present inventions are used for treatment or prevention of viral hepatitis or HIV infections, etc.

In another aspect of the present invention the cyclic peptides according to general formula I are particularly useful for the treatment of proliferative disorders like cancer or tumors. In particular, the cancer is any one of multiple myeloma, chronic lymphocytic leukemia, low-grade lymphoma, Kaposi's sarcoma, chronic myelogenous leukemia, renal-cell carcinoma, cervical carcinoma, urinary bladder tumors and ovarian cancer.

In still another embodiment, the present invention relates the use of the cyclic peptides according to the present invention for the treatment or prevention of neurologic disorders, in particular, of multiple sclerosis or Alzheimer's disease.

The inventors found that the cyclic peptides according to general formula I are able to enhance the activity of interferon type I, in particular of interferon alpha and interferon beta; type I interferon is an important substance involved in immune response of the innate and adaptive immune system. In particular, enhancing type I interferon response allows to modulate the immune response. Thus, the claimed compounds display an immunomodulatory activity.

That is, the cyclic peptides having the general formula I are useful for inhibiting replication of a virus in cells infected with the virus and/or reducing the number of copies of the viruses in the cells infected with the virus. Further, the compounds of the general formula I are able to prevent infection, in particular, to prevent viral infection of cells in a subject, in particular, in animals and more preferably in humans.

Further, the cyclic peptides of the general formula I are particularly useful to prevent or treat cancer or other diseases or disorders involving proliferation of cells, namely proliferative disorders, since the cyclic peptides are able to inhibit the proliferation of cells in particular of tumour cells.

As mentioned above, the present inventors found that the cyclic peptide of the general formula I induces interferon type I expression and/or enhance and/or support type I interferon mediated metabolic reactions, like anti-viral effects, enhancement of immune reaction, anti-proliverative effects, etc.

Furthermore, it appears that even if cells do not express type I interferon receptor an anti-viral effect can be observed after applying the cyclic peptides according to the present invention.

Thus, administration of the cyclic peptides of the general formula I alone or together with interferon gamma type I and/or other active ingredients are useful in the prevention or treatment of e.g. infectious diseases or to enhance and/or support the body defense in inflammation reactions. This may be particular useful in subjects suffering from immunodeficiency or subjects being immunosuppressed due to hereditary defects or due to therapeutic regimens.

In particular, the combination of the cyclic peptides of the general formula I or conjugates thereof, or salts or solvates thereof with interferon type I allows a dramatic reduction of the dosage of interferon type I and, consequently, enables to reduce the side-effects associated therewith, like receptor cross-reactivity, toxicity, etc as outlined above.

The cyclic peptides according to the present invention are known to have low toxicity. Thus, said compounds are extremely useful for enhancing immune reaction, in particular, when co-administered with interferon type I.

That is, the cyclic peptides, i.e. the vioprolides, are extremely useful for enhancing interferon type I treatment. Thus, the use of the cyclic peptide in pharmaceutical preparations and in the treatment or prevention of diseases where interferon type I treatment is administered, is particularly envisaged.

Therefore, in another embodiment of the present invention, pharmaceutically compositions are provided comprising an effective amount of a cyclic peptide having the general formula I and an effective amount of interferon type I optionally together with a pharmaceutically acceptable excipient or diluent.

In a preferred embodiment, the interferon type I is interferon alpha (IFN alpha). In another preferred embodiment the interferon type I is interferon beta (IFN beta).

Said pharmaceutical composition is particular useful for treating infectious diseases, neurological disorders and cancer, e.g. specific types of leukemia.

Today interferon type I is used for the treatment of hepatitis C and other viral hepatitis. Further, interferon alpha is approved for the treatment of chronic myelogenous leukemia.

Furthermore, interferon beta is widely used for the treatment and control of the neurological disorder multiple sclerosis. In addition, interferon type I has been shown to be useful to prevent and treat viral respiratory diseases such as cold and flu. Hence, pharmaceutical compositions comprising the cyclic peptides of general formula I beside the active ingredient interferon type I, are particular preferred embodiments of the present invention useful for the treatment or prevention of the diseases, disorders or conditions mentioned above.

Thus, in another embodiment of the present invention, methods for the treatment or prevention of infectious disease, in particular, of viral infections are provided. The present invention further relates to methods for the treatment or prevention of infectious diseases, in particular of viral, bacterial or parasite infections whereby the replication or reproduction of virus, bacteria or parasites is inhibited. In another aspect, the method is directed to the treatment or prevention of infectious diseases by reducing the number of copies of the bacteria, virus and/or parasites. In a further aspect, the present invention is directed to a method for the treatment or prevention of infectious diseases, in particular viral infection by protecting the subject from viral, bacterial or parasite infections.

The method according to the present invention is characterized in comprising the step of administering the cyclic peptide of the general formula I systemically or locally. In a preferred embodiment the method comprise the local administration of the cyclic peptide of the general formula I for local enhancement of the activity of interferon type I in particular of interferon alpha or interferon beta.

For example, interferon type I may be administered systemically while the cyclic peptides according to the present invention are administered locally. This allows the site specific support and/or enhancement of interferon type I, e.g. in the central nervous system, the brain or other specific sites of action. The separate administration of the two compounds which may be sequentially may be applied in the treatment of multiple sclerosis or in case of viral infections of specific tissues or organs. Of course, systemic administration of the cyclic peptides and local administration of interferon type I is possible.

Another embodiment of the present invention relates to the use of the cyclic peptide having the general formula I or conjugates thereof, and salts or solvates thereof as adjuvants for therapeutic or prophylactic vaccination adjuvants are characterized in being substances which are added and/or co-formulated in an immunization to the active antigen, e.g. the substance which provokes the desired immune response, in order to enhance or elicit or modulate the humoral and/or cell mediated (cellular) immune response against the active antigen. In the present case, it is preferred that the cyclic peptide having the general formula I or conjugate thereof, and salts or solvates thereof is used to direct the immune response to a specific Th type, namely Th1 type. That means, using the cyclic peptide having the general formula I or conjugates thereof, and salts or solvates thereof as adjuvants for therapeutic or prophylactic vaccination allows to direct the immune response elicited during the vaccination to a more Th1 phenotype by enhancement of differentiation of naive T-cells to a Th1 phenotype and/or suppression of differentiation of naïve T-cells to a Th2 phenotype. This might be extremely useful for example in vaccines useful to desensitize subjects since typically an IgE response occurring in allergic responses is based on the formation of IL-4 producing Th helper cells, i.e. Th2 helper type T cells.

The cyclic peptide may be formulated into the vaccine according to known methods.

As used herein, the term “conjugate” refers to compounds comprising a conjugate moiety and a compound moiety. The conjugate moiety aims to increase the applicability of the residual compound. The conjugate moiety of the conjugate according to the present invention is a covalently bonded, physiologically tolerated conjugate moiety, which is suitable for converting the cyclic peptides into an even more water-soluble form. For example, the conjugate moiety can be a polymer, a dextran, a sugar, a polyvinylpyrrolidone, an alginate, a pectin or collagen. The conjugate moiety is characterized in that it provides good water solubility and is not immunogenic.

The conjugate moiety of the cyclic peptide conjugate claimed herein, is in a preferred embodiment, a conjugate moiety containing at least one polyalkylene glycol unit of the formula:

X₁—[(CHR₁)ₓ—O]ₙ-(Z)_{y}-
where
X₁ is hydrogen or a hydrocarbon which may contain heteroatom(s);
Z is a divalent linkage group, such as C═O or CHR₁;
R₁ is independently any one of hydrogen, OH, OR₂ or CO—R₃;
R₂ is independently any one of hydrogen or C₁-C₆ alkyl;
R₃ is independently any one of hydrogen, OH, OR₂ or NR₄R₅;
R₄ and R₅ are independently any one of hydrogen or hydrocarbon which may contain heteroatom(s) and which may form a ring;
n is an integer of 1 to 100;
x is independently an integer of 1 to 10;
y is an integer of 0 to 10.

Preferably, n is an integer of 2 to 50, like 2 to 10, in particular 3 to 5.

In another embodiment, x is preferred an integer of 2, 3, or 4, in particular 2.

y is preferred an integer of 1 to 5, in particular, 1 to 3, in another preferred embodiment, y is 0.

X₁ is preferentially OR₆, N(R₆)₂, SR₆ or COOR₆, wherein each R₆ is individually hydrogen, benzyl or C₁-C₆ alkyl, preferably a C₁-C₆ alkoxy group, like a methoxy, ethoxy or propoxy group.

R₁ is preferably a hydrogen atom.

Thus, the polyalkylene glycol unit mentioned above may preferably contain subunits of ethylene glycol, propylene glycol or butylene glycol or combinations thereof. The chain length of each of the polyalkylene glycol units may be in the range of 1 to 100 subunits, preferably, 2 to 50 subunits, like 2 to 10 subunits, particularly in the range of 3 to 5 subunits.

Particularly preferred is the conjugate moiety a methoxypolyalkyleneglycol-carbonyl-residue wherein the alkylene moiety is an ethylene or propylene moiety.

Hence, preferably the conjugates are in a pegylated form to increase the solubility in hydrophilic solvents and hydrophilic environment. Furthermore, the conjugate moiety allows protecting the compound moiety against enzymatic degradation, structural modification due to change of the pH, mechanical removal, etc. Thus, primarily the stability of the compound is increased. Another beneficial effect of conjugation is to increase the retention time in the individual, e.g. to delay the renal excretion, while being well-tolerated, e.g. being non-immunogenic, by said organism.

Specifically, the conjugate moiety comprises at least two chains having polyalkylene glycol units. That is, the conjugate may be a branched compound wherein each arm contains a polyalkylene glycol unit. Particularly preferred are conjugate moieties wherein the polyalkylene glycol unit is a polyethylene, polypropylene or polybutylene glycol unit.

As used herein, the term “pegylated” refers to the conjugation of a compound moiety with conjugate moiety(ies) containing at least one polyalkylene unit. In particular, the term pegylated refers to the conjugation of the compound moiety with a conjugate moiety having at least on polyethylene glycol unit.

### Formulations and Routes of Administration

Therapeutic formulations of the polypeptide or conjugate of the invention are typically administered in a composition that includes one or more pharmaceutically acceptable carriers or excipients. Such pharmaceutical compositions may be prepared in a manner known per se in the art to result in a polypeptide pharmaceutical that is sufficiently storage-stable and is suitable for administration to humans or animals.

### Drug Form

The cyclic peptide or conjugate of the invention can be used “as is” and/or in a salt form thereof. These salts or complexes may by present as a crystalline and/or amorphous structure. The pharmaceutical composition for use in connection with the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Excipients

“Pharmaceutical acceptable” means a carrier or excipient that at the dosages and concentrations employed does not cause any untoward effects in the patients to whom it is administered. Such pharmaceutically acceptable carriers and excipients are well known in the art (see Remington's Pharmaceutical Sciences, 18^{th} edition, A. R. Gennaro, Ed., Mack, Publishing Company (1990); Pharmaceutical Formulation Development of Peptides and Proteins, S. Frokjaer and L. Hovgaard, Eds., Taylor & Francis (2000); and Handbook of Pharmaceutical Excipients, 3^{rd} edition, A. Kibbe, Ed., Pharmaceutical Press (2000)).

The term “carrier” or “exceipient” refers to a carrier, diluent, adjuvant, excipient, or vehicle with which the active ingredient is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium, carbonate, etc. Such compositions will contain a therapeutically effective amount of the aforementioned compounds or conjugates thereof, and salts or solvates thereof, preferably in purified form, together with a suitable amount of carrier so as to provide the form for proper administration to the patient. The formulation should suit the mode of administration.

Typically, pharmaceutically or therapeutically acceptable carrier is a carrier medium which does not interfere with the effectiveness of the biological activity of the active ingredients and which is not toxic to the host or patient.

### Mix of Drugs

The pharmaceutical composition of the invention may be administered alone or in conjunction with other therapeutic agents. Ribavirin, for example, is currently co-administered with IFN-alpha and has been shown to increase efficacy in HCV treatment. A variety of small molecules are being developed against both viral targets (viral protease, viral polymerase, assembly of viral replication complexes) and host targets (host proteases required for viral processing, host kinases required for phosphorylation of viral targets such as NS5A and inhibitors of host factors required to efficiently utilize the viral IRES). Other cytokines may be co-administered, such as IL-12, IL-23, IL-27 or IFN-gamma. These agents may be incorporated as part of the same pharmaceutical composition or may be administered separately from the polypeptide or conjugate of the invention, either concurrently or in accordance with another treatment schedule. In addition, the polypeptide, conjugate or pharmaceutical composition of the invention may be used as an adjuvant to other therapies.

For example, one of the active compounds may be formulated to be administered locally while the other active ingredient may be formulated to be administered systemically. For example, in systemic treatment with interferon type I, the cyclic peptide may be administered locally to support and/or enhance the treatment at specific sites of the body.

### Patients

A “patient” for the purposes of the present invention is a subject or individual as defined above. It includes both humans and other mammals. Thus, the methods are applicable to both human therapy and veterinary applications.

### Types of Composition and Administration Route

The pharmaceutical composition comprising the cyclic peptide or conjugate of the invention may be formulated in a variety of forms, e.g. as a liquid, gel, lyophilized, or as a compressed solid. The preferred form will depend upon the particular indication being treated and will be apparent to one skilled in the art.

The administration of the formulations of the present invention can be performed in a variety of ways, including, but not limited to, orally, subcutaneously, intravenously, intracerebrally, intranasally, transdermally, intraperitoneally, intramuscularly, intrapulmonary, vaginally, rectally, intraocularly, or in any other acceptable manner. The formulations can be administered continuously by infusion, although bolus injection is acceptable, using techniques well known in the art, such as pumps (e.g. subcutaneous osmotic pumps) or implantation. In some instances the formulations may be directly applied as a solution or spray.

### Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance to stability of the active compound contained in the compositions under a wider variety or storage conditions as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

Parenterals may be prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g. tartaric acid-sodium tartrate mixture, tartaric acid-potassium tartrate mixture, tartaric acid-sodium hydroxide mixture, etc.), fumarate buffers (e.g. fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g. oxalic acid-sodium oxalate mixture, oxalic acid-sodium hydroxide mixture, oxalic acid-potassium oxalate mixture, etc.), lactate buffers (e.g. lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerine, erythritol, arabitol, xylitol, sorbitol and mannitol. Polyhydric alcohols can be present in an amount between 0.1% and 25% by weight, typically 1% to 5% taking into account the relative amounts of the other ingredients.

Stabilizers refer to a broad category of excipients which can range in function from a bulking agent to an additive which solubilizers the therapeutic agent or helps to prevent denaturation or adherence to the container wall. Typical stabilizers can be polyhydric sugar alcohols (enumerated above); amino acids such as arginine, lysine, glycine, glutamine, asparagines, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate, low molecular weight polypeptides (i.e. <10 residues); proteins such as human serum albumin, bovine serum albumin, gelatine or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80 etc), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g. ascorbic acid, methionine, vitamin E) and cosolvents.

The active ingredient may also be entrapped in microcapsules prepared, for example, by coascervation techniques or by interfacial polymerization, for example hydroxymethylcellulose, gelatine or poly-(methylmethacrylate) microcapsules, in colloidal drug delivery systems (for example liposomes, albumin microspheres, microemulsions, nano-particles and nanocapsules) or in macroemulsions. Such techniques are discloses in Remington's Pharmaceutical Sciences, supra.

Parenteral formulations to be used for in vivo administration must be sterile. This is readily accomplished, for example, by filtration through sterile filtration membranes.

### Sustained Release Preparations

Suitable examples for sustained-release preparations include semi-permeable matrices of solid hydrophobic polymers containing the cyclic peptide or conjugate, the matrices having a suitable form such as a film or microcapsules. Examples of sustained-release matrices include polyesters, hydrogels (for example, poly(2-hydroxyethyl-methacrylate) or poly(vinylalcohol), polylactides, copolymers of L-glutamic acid and ethyl-L-glutamate, non-degradable ethylene-vinyl acetate, degradable lactic acid-glycolic acid copolymers such as the ProLease® technology or Lupron Depot®) (injectable microspheres composed of lactic acid-glycolic acid copolymer and leuprolide acetate), and poly-D-(-)-3-hydroxybutyric acid. While polymers such as ethylene-vinyl acetate and lactic-glycolic acid enable release of molecules for long periods such as up to or over 100 days, certain hydrogels release proteins for shorter time periods. When encapsulated cyclic peptide remain in the body for a long time, they may denature or aggregates as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### Oral Administration

For oral administration, the pharmaceutical composition may be in solid or liquid form, e.g. in the form of a capsule, tablet, suspension, emulsion or solution. The pharmaceutical composition is preferably made in the form of a dosage unit containing a given amount of the active ingredient. A suitable daily dose for a human or other mammal may vary widely depending on the condition of the patient and other factors, but can be determined by persons skilled in the art using routine methods.

Solid dosage forms for oral administration may include capsules, tablets, suppositories, powders and granules. In such solid dosage forms, the active compound may be admixed with at least one inert diluent such as sucrose, lactose, or starch. Such dosage forms may also comprise, as is normal practice, additional substances, e.g. lubricating agents such as magnesium stearate. In the case of capsules, tablets and pills, the dosage forms may also comprise buffering agents. Tablets and pills can additionally be prepared with enteric coatings.

The cyclic peptides or conjugates may be admixed with adjuvants such as lactose, sucrose, starch powder, cellulose esters of alkanoic acids, stearic acid, talc, magnesium, stearate, magnesium oxide, sodium and calcium salts of phosphoric and sulphuric acids, acacia, gelatine, sodium alginate, polyvinyl-pyrrolidine, and/or polyvinyl alcohol, and tableted or encapsulated for conventional administration. Alternatively, they may be dissolved in saline, water, polyethylene glycol, propylene glycol, ethanol, oils (such as corn oil, peanut oil, cottonseed oil or sesame oil), tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be subjected to conventional pharmaceutical operations such as sterilization and/or may contain conventional adjuvants such as preservatives, stabilizers, wetting agents, emulsifiers, buffers, fillers etc., e.g. as disclosed elsewhere herein.

Liquid dosage forms for oral administration may include pharmaceutically acceptable emulsions, solutions, suspensions, syrups and elixirs containing inert diluents commonly used in the art, such as water. Such compositions may also comprise adjuvants such as wetting agents, sweeteners, flavouring agents and perfuming agents.

### Pulmonary Delivery

Formulations suitable for use with a nebulizer, either jet or ultrasonic, will typically comprise the cyclic peptide or conjugate dissolved in water at a concentration of e.g. about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles of the invention are described in WO 94/20069, U.S. Pat. No. 5,915,378, U.S. Pat. No. 5,960,792, U.S. Pat. No. 5,957,124, U.S. Pat. No. 5,934,272, U.S. Pat. No. 5,915,378, U.S. Pat. No. 5,855,564, U.S. Pat. No. 5,826,570 and U.S. Pat. No. 5,522,385 which are hereby incorporated by reference.

Formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparations if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. An example of a commercially available metered dose inhaler suitable for use in the present invention is the Ventolin metered dose inhaler, manufactured by Glaxo Inc., Research Triangle Park, N.C., USA.

Formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 an 5 micrometers, most preferably of between 1.5 and 3.5 micrometers. An example of a powder inhaler suitable for use in accordance with the teachings herein is the Spinhaler powder, manufactured by Fisons Corp., Bedford, Mass., USA.

The powders for these devices may be generated and/or delivered by methods disclosed in U.S. Pat. No. 5,997,848, U.S. Pat. No. 5,993,783, U.S. Pat. No. 5,985,248, U.S. Pat. No. 5,976,574, U.S. Pat. No. 5,922,354, U.S. Pat. No. 5,785,049 and U.S. Pat. No. 5,654,007.

Mechanical devices designed for pulmonary delivery of therapeutic products, include but are not limited to nebulizers, metered dose inhalers, and powder inhalers, all of which are familiar to those of skill in the art. Specific examples of commercially available devices suitable for the practice of this invention are the Ultravent nebulizer, manufactured by Mallinckrodt, Inc., St. Louis, Mo., USA; the Acorn II nebulizer, manufactured by Marquest Medical Products, Englewood, Colo., USA; the Ventolin metered dose inhaler, manufactured by Glaxo Inc., Research Triangle Park, N.C., USA; the Spinhaler powder inhaler, manufactured by Fisons Corp., Bedford, Mass., USA the “standing cloud” device of Nektar Therapeutics, Inc., San Carlos, Calif., USA; the AIR inhaler manufactured by Alkernes, Cambridge, Mass., USA; and AERx pulmonary drug delivery system manufactured by Aradigm Corporation, Hayward, Calif., USA.

Furthermore, the interferon type I may be in form of the pure substance, in form of a pharmaceutically acceptable salt or solvate thereof or in form of a conjugate. In particular, interferon type I in a pegylated form can be used.

Thus, according to the present invention, it is possible to reduce the dosage of type I interferon administration. Hence, the clinical utility of type I interferon is broadened and the risk of elevating negative side-effects, including receptor cross-reactivity or toxicity is decreased.

In order that the invention may be readily understood and put into practically effect, invention will now be described by way of the following non-limiting examples.

### EXAMPLES

### Example 1

### Method for Determining Type I Interferon Activity

The method to determine the biological activity of murine type I interferon is based on the cell base-virus free system as disclosed in Bollati-Fogolin, M. and Müller W., J. Immunol. Meth., 2005, 306, 169-175, which is herein incorporated by reference.

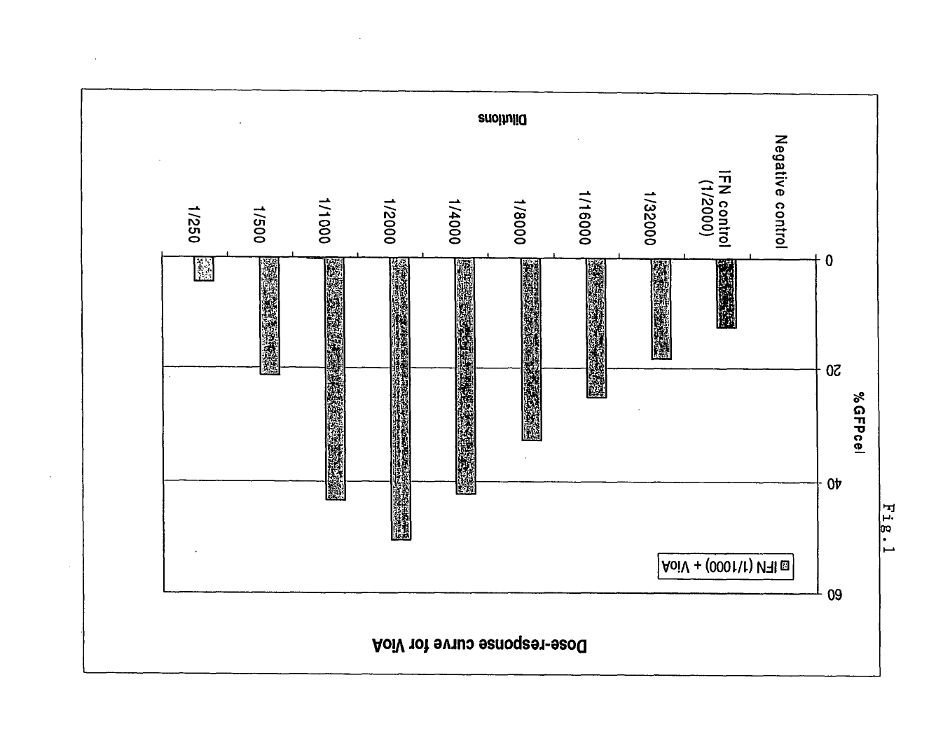

In short, fibroblast cell lines, MXRage cells carrying the MxCre gene and a promotorless eGFP gene where used. The preparation of said cells is described in the publication Bollati-Fogolin, supra. Said cells were incubated with either murine interferon alpha 11 alone or in combination with any one of the vioprolides A to D in an amount of 10 nm/ml. The amount of vioprolides was tested before in a dose response curve as shown in FIG. 1.

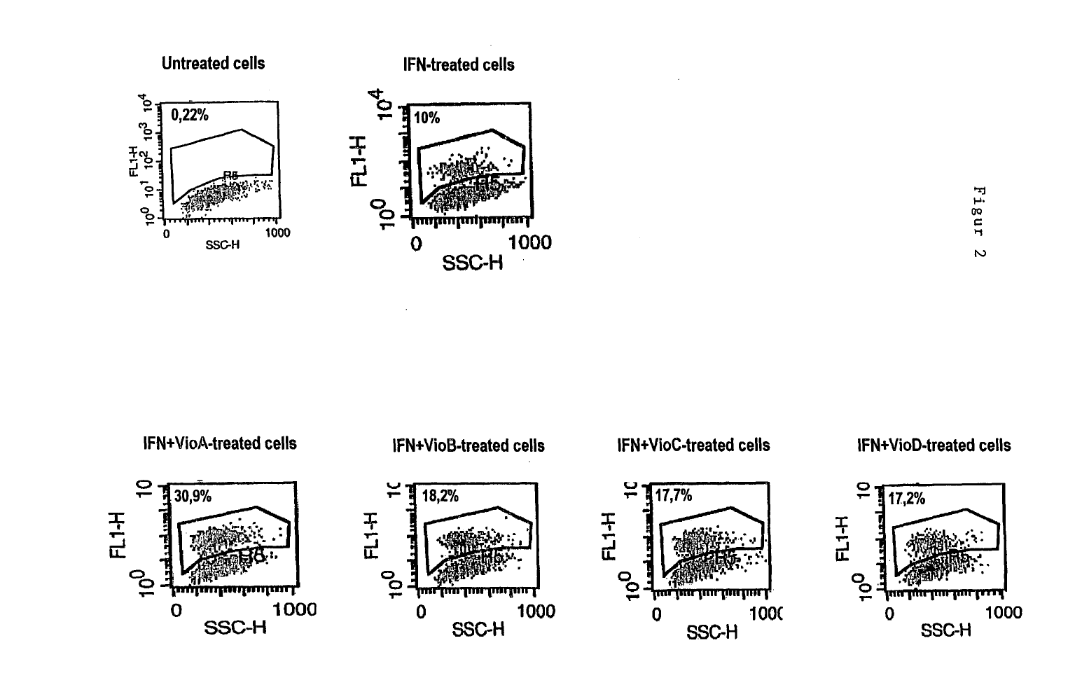

After 72 hours cultivation the cells were trypsinated and analyzed by flow cytometry FIG. 2 outlines a representative result obtained wherein in the upper part untreated cells and interferon gamma treated cells are shown. The lower panel shows the data obtained when incubating said cells with a combination of interferon alpha 11 and the vioprolide as indicated. As can be derived from FIG. 2 the addition of any one the vioprolides A to D increases the number of eGFP positive cells (green fluorescence) when analyzed by flow cytometry. Thus, FIG. 2 demonstrates that vioprolides enhance the activity of interferon type I.

### Example 2

### Analysis of Vioprolide Concentration for Interferon Type I Enhancement

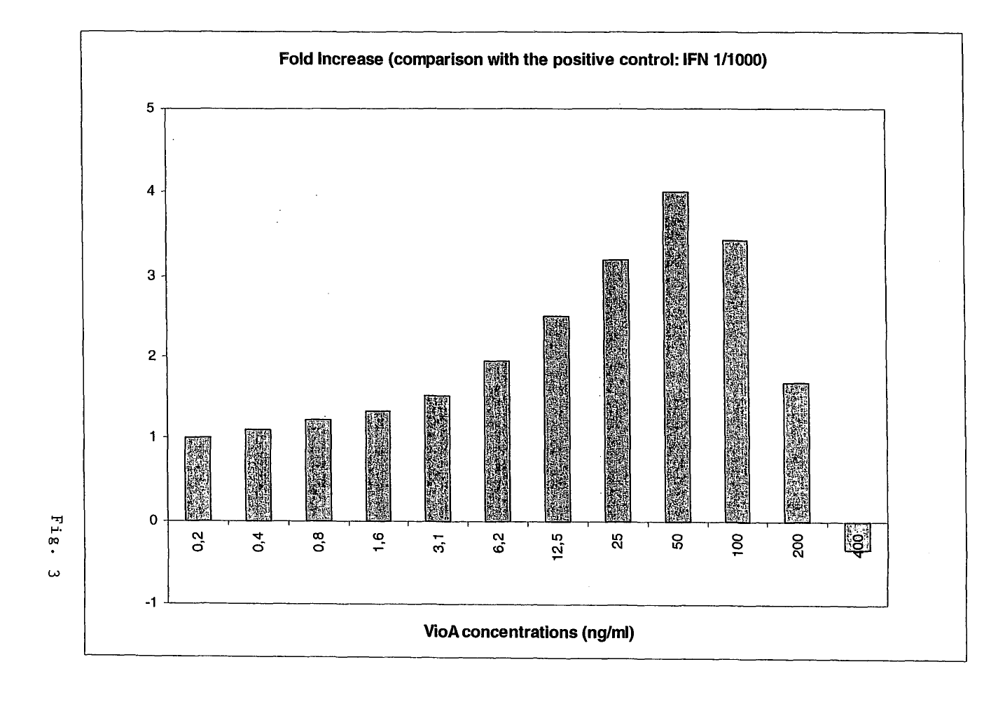

To study the influence of various concentrations of vioprolides on the increase of interferon type I induction, various dilutions of vioprolides were tested in the above mentioned test system. Serial two-fold solutions were tested for each of the vioprolides. FIG. 3 shows as an example the results obtained for vioprolide A. The vioprolide A concentration tested was in the range of 0.2 to 400 ng/ml. FIG. 3 demonstrates that at best an 4-fold increase can be achieved when using vioprolide A in combination with interferon type I compared to the use of interferon type I alone.

### Example 3

### Vioprolide A Effect on the Alpha Interferon and the Beta Interferon Standard Curve

With the test system as described above the influence of vioprolide A on alpha and beta interferon standard curves have been determined. The cells in the test system were incubated with either various dilutions of interferon type I alone or in combination with vioprolide A in an amount of 50 ng/ml.

The analysis of GFP positive cells were performed as described above.

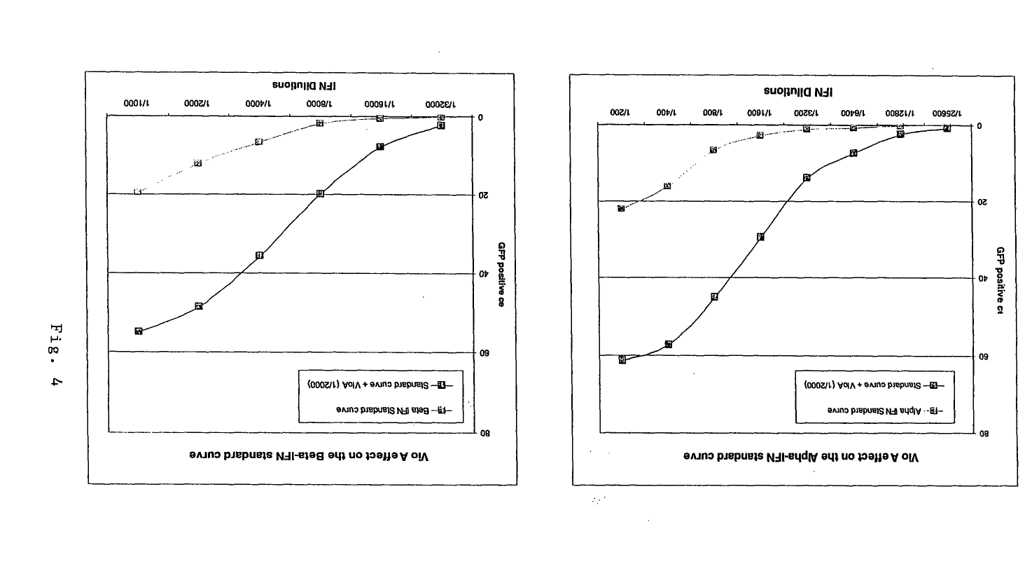

As can be derived from FIG. 4 vioprolide A provides a remarkable increase of GFP positive cells when incubated with interferon alpha and interferon beta, respectively. It is demonstrated that vioprolide A is able to enhance interferon type I activity. For example, as can be derived from FIG. 4 left panel using vioprolide A together with interferon alpha allows to reduce the amount of interferon alpha about 10 fold to obtain the same percent of GFP positive cells. The same is true for IFN beta as shown in the right panel of FIG. 4.

### Example 4

### Vioprolide A Antiviral Effect

Using standards methods, the vioprolide A antiviral effect was investigated using two different types of virus, Vesicular stomalitis virus (VSV, ssRNA negative) and Herpes simplex virus strain 1 (HSV-1, dsDNA) in MDBK and Vero cells, respectively.

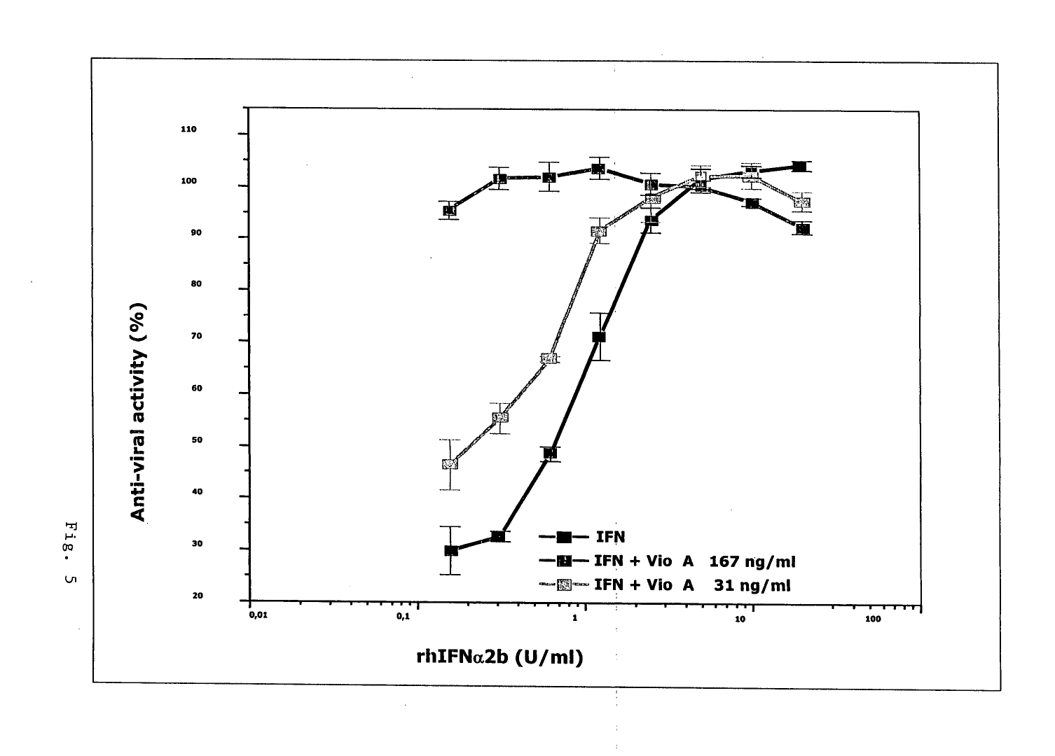

Different concentrations of rhIFN-α2b alone and in combination with 31 and 167 ng/ml of vioprolide A, respectively, were assayed, see FIG. 5. As demonstrated in FIG. 5, vioprolide A is able to enhance the anti-viral activity of IFN on VSV.

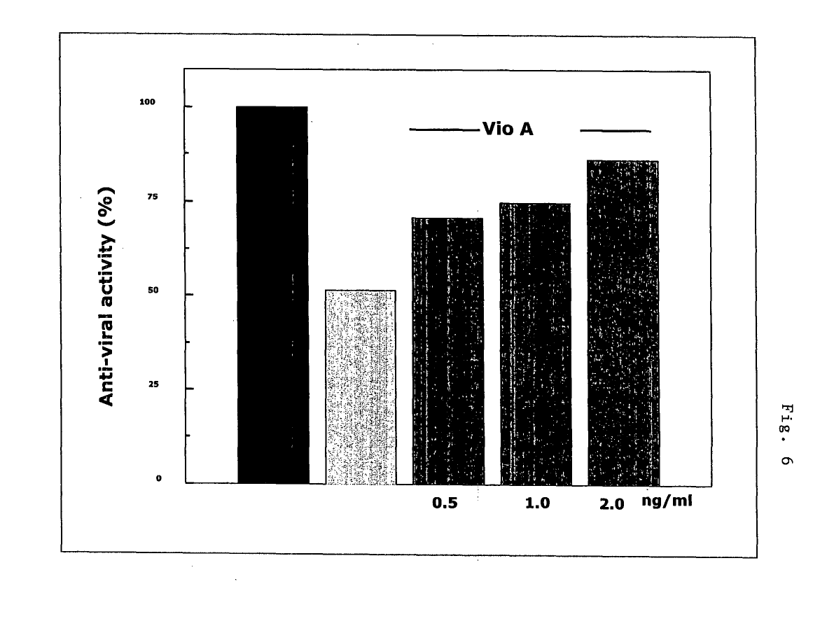

Further, vioprolide A display an anti-viral activity on HSV-1 is shown in FIG. 6. Different concentrations of vioprolide A were assayed in a Vero cells system. Increasing amounts of vioprolide A demonstrates an anti-viral activity.

### Example 5

### Vioprolide A Antiproliferative Effect

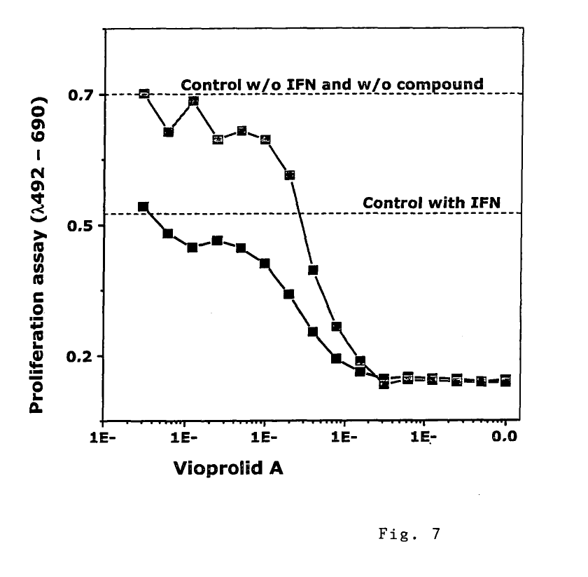

Further, the antiproliferative effect of vioprolide A has been determined. Using standard assays, the antiproliferative activity was evaluated with human cervical carcinoma derived cell line WISH. In the presence of rhIFN-α2b, vioprolide A shows an additive effect on cell growth inhibition, see FIG. 7.

Thus, not only anti-viral activity but also antiproliferative activity is demonstrated for vioprolides. In particular, vioprolides show synergistic effects with type I interferon.

1. A use of a cyclic peptide having the general formula I 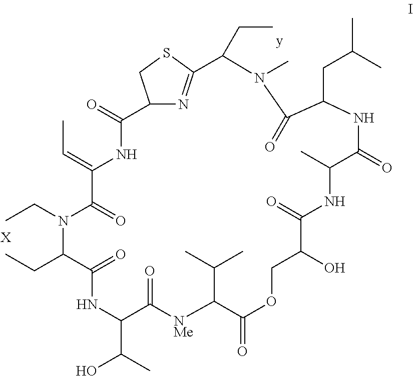
wherein X represents a methylene or an ethylene group and Y is an ethylene or a methylmethylene group,
or conjugates thereof, and salts or solvates thereof,
for the preparation of a pharmaceutical for the treatment or prevention of infectious diseases, septic shock, cancer, tumours or other proliferative disorders, autoimmune diseases, neurological disorders, allergies, or chronic or acute inflammatory processes.

2. The use according to claim 1 wherein said disease is an infectious disease.

3. The use according to claim 2 wherein said infectious diseases is a viral infection.

4. The use according to claim 3 wherein said viral infectious is a viral hepatitis in particular Hepatitis B, Hepatitis C, or HIV infection.

5. The use according to claim 1 wherein said disease is cancer or tumors.

6. The use according to claim 5 wherein said cancer is selected from the group consisting of multiple myeloma, chronic lymphocytic leukemia, low-grade lymphoma, Kaposi's sarcoma, chronic myelogenous leukemia, renal-cell carcinoma, cervical carcinoma, urinary bladder tumors and ovarian cancers.

7. The use according to claim 1 wherein said disease are neurological disorders, preferably multiple sclerosis or Alzheimer's disease.

8. The use of a cyclic peptide having the general formula I 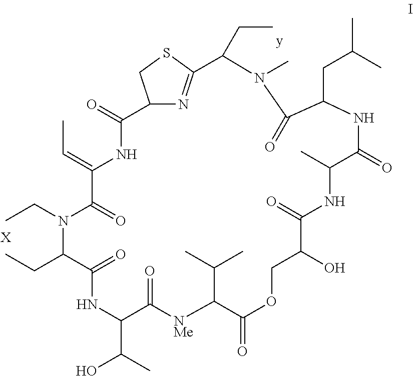
wherein X represents a methylene or an ethylene group and Y is an ethylene or a methylmethylene group,
or conjugates thereof, and salts or solvates thereof,
for the manufacture of a pharmaceutical for inhibiting replication of a virus in cells infected with the virus and/or reducing the number of copies of the virus in the cells infected with the virus and/or preventing viral infection of cells.

9. The use of a cyclic peptide of the general formula I 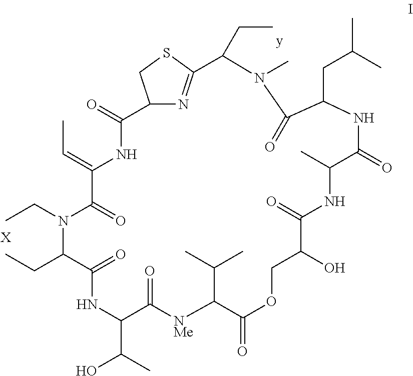
wherein X represents a methylene or an ethylene group and Y is an ethylene or a methylmethylene group,
or conjugates thereof, and salts or solvates thereof,
for the manufacture of a pharmaceutical for inhibiting proliferation of cells, in particular of tumor cells.

10. Pharmaceutical composition comprising an effective amount of a cyclic peptide having the general formula I and an effective amount of interferon type I and, optionally, a pharmaceutically acceptable excipient.

11. The pharmaceutical composition according to claim 10, wherein said interferon type I is interferon alpha.

12. The pharmaceutical composition according to claim 10, further comprising an additional anti-viral drug.

13. The pharmaceutical composition according to claim 10 wherein said interferon type I is interferon beta.

14. The use of an effective amount of a cyclic peptide having the general formula I 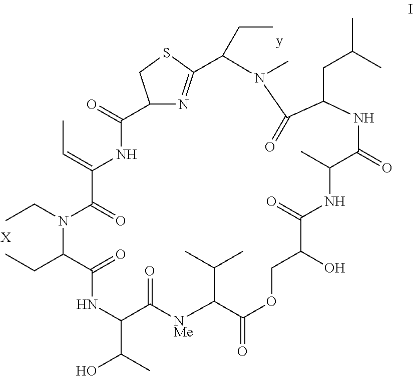
wherein X represents a methylene or an ethylene group and Y is an ethylene or a methylmethylene group,
or conjugates thereof, and salts or solvates thereof,
and an effective amount of type I interferon for the preparation of a pharmaceutical for the treatment or prevention of infectious diseases, septic shock, cancer, tumours or other proliferative disorders, autoimmune diseases, neurological disorders, allergies or chronic or acute inflammatory processes.

15. The use according to claim 14 wherein said disease is an infectious disease.

16. The use according to claim 15 wherein said infectious diseases is a viral infection.

17. The use according to claim 16 wherein said viral infectious is a viral hepatitis or HIV infection.

18. The use according to claim 14 wherein said disease is cancer or tumors.

19. The use according to claim 18 wherein said cancer is selected from the group consisting of multiple myeloma, chronic lymphocytic leukemia, low-grade lymphoma, Kaposi's sarcoma, chronic myelogenous leukemia,
renal-cell carcinoma, cervical carcinoma, urinary bladder tumors and ovarian cancers.

20. The use according to claim 14 wherein said disease are neurological disorders, preferably multiple sclerosis or Alzheimer's disease.

21. Method for the treatment or prevention of viral infection, in particular, of inhibiting replication of a virus in cells infected with the virus and/or reducing the number of copies of the virus in the cells infected with the virus and/or preventing viral infection comprising administering a cyclic peptide of the general formula I.

22. A method for the treatment or prevention of a disease, disorder or condition selected from the group consisting of infectious diseases, septic shock, cancer, tumours or other proliferative disorders, autoimmune diseases, neurological disorders, allergies or chronic or acute processes comprising administering a cyclic peptide of the general formula 1.

23. A method according to claim 22 for the treatment or prevention of a disease, disorder or condition, wherein said disease is selected from the group consisting of HCV, HBV, HIV, leukaemia, carcinoma and multiple sclerosis.

24. A method according to claim 21 further comprising the step of simultaneously, separately or sequentially administering type I interferon, in particular of interferon alpha or interferon beta.

25. Use of a cyclic peptide having the general formula I or conjugates thereof, and salts or solvates thereof, as adjuvants for therapeutic or prophylactic vaccination.
